(19) United States
(12) Patent Application Publication
(10) Pub. No.: US 2015/0044154 A1
(43) Pub. Date: Feb. 12, 2015

(54) COMPOSITION FOR THE TREATMENT OF KERATINOUS FIBERS COMPRISING AT LEAST ONE FIXING POLYMER AND AT LEAST ONE COMPOUND OF THE CERAMIDE TYPE AND METHODS FOR USING THE COMPOSITION

(71) Applicant: L'OREAL, Paris (FR)

(72) Inventors: Christine DUPUIS, Paris (FR); Claude DUBIEF, Le Chesnay (FR); Isabelle CRETOIS, Paris (FR); Damarys BRAIDA-VALERIO, Paris (FR)

(21) Appl. No.: 14/309,269

(22) Filed: Jun. 19, 2014

Related U.S. Application Data
(63) Continuation of application No. 12/978,862, filed on Dec. 27, 2010, now abandoned.
Application No. 12/978,862 is a continuation of application No. 08/740,142, filed on Oct. 22, 1996, now abandoned.

(30) Foreign Application Priority Data
Oct. 23, 1995 (FR) 95-12448

(51) Int. Cl. A61K 8/68; A61Q 5/06; A61K 8/81

(52) U.S. Cl. CPC A61K 8/68; A61K 8/8164; A61K 8/8152; A61Q 5/06; A61K 2800/40
USPC 424/70.11

(57) ABSTRACT

The invention relates to a cosmetic composition for the treatment of keratinous fibres, in particular human hair, comprising at least one anionic, nonionic, amphoteric or zwitterionic fixing polymer and at least one compound of the ceramide type as well as to the method of treatment using this composition.

The present invention relates to a cosmetic composition for the treatment of keratinous fibers such as hair, comprising at least one fixing polymer and at least one component of the ceramide type, as well as to the method of nontherapeutic treatment with the aid of this composition.

Compositions for holding or shaping the hair, containing, in their formulation, hair-styling polymers (fixing polymers), generally have the disadvantage of making difficult the disentangling, restyling or brushing of the hair, in particular during blow-drying.

During blow-drying, the hair is damaged by the heat from the dryer and the passing of the brush through the hair in order to shape the hair

Many hair strands are thus broken during blow-drying. Compositions are therefore being sought which make it possible to protect the hair from this breaking-off during these aggressions.

The use of silicone-containing derivatives in combination with fixing polymers is known in the preparation of cosmetic compositions for holding the hairstyle. It has been observed that these silicone-containing derivatives improve the disentangling, softness and sheen properties of hair treated with these compositions. However, on the one hand, silicone-containing derivatives are not favorable to the hair-styling properties of compositions containing fixing polymers and, on the other hand, the protecting effect against the breaking-off of the hair is not yet satisfactory.

Now, the inventors have discovered, surprisingly, that by using compositions containing a fixing polymer in combination with compounds of the ceramide type, a very good protecting effect was obtained against the breaking-off of the hair, in particular during blow-drying, while having excellent hair-styling properties.

The hair-styling properties are of the same level or even superior to those of a composition containing only the fixing polymer. In particular, the fixing power, the behavior over time and the hair volume are very good.

This discovery forms the basis of the present invention.

The subject of the invention is therefore a nondetergent cosmetic composition intended for the treatment of keratinous fibers such as the hair, characterized in that it contains, in a cosmetically acceptable medium, at least one fixing polymer and at least one compound of the ceramide type, the compositions not containing any vinylpyrrolidone polymer and/or cationic polymer containing primary, secondary or tertiary amine or quaternary ammonium groups in the principal chain and having a viscosity at 1% by weight of active substance in water of less than 15 mPa·s.

The subject of the invention is also the use of the composition defined above for protecting the hair during blow-drying.

These compositions also make it possible to improve the cosmetic properties, in particular the softness and sleekness, of the hair.

Fixing power of the composition denotes the capacity of the latter to give the hair a cohesion such that the initial shaping of the hairstyle is preserved. Fixing polymer is understood to mean any polymer whose function is to temporarily fix the shape of the hairstyle.

The term nondetergent means that the composition does not make it possible to eliminate from a solid medium such as, for example, the hair, the dirt adhering thereto by dispersing or dissolving it. Preferably, the compositions according to the invention comprise less than 4% by weight relative to the total weight of the composition of anionic or amphoteric detergent surfactants.

Vinylpyrrolidone polymer denotes the polymers containing at least the vinylpyrrolidone monomer.

According to the present invention, ceramide is understood to mean the natural or synthetic ceramides and/or glycoceramides and/or pseudoceramides and/or neoceramides.

Ceramides are, for example, described in the patent applications DE4,424,530; DE4,424,533; DE4,402,929; DE4,420,736; WO95/23807; WO94/07844; EP-A-0,646,572; WO95/16665; FR-2,673,179; EP-A-0,227,994 and WO94/07844; WO94/24097; WO94/10131, all of which are incorporated herein by reference.

The compounds of the ceramide type which can be used according to the present invention preferably correspond to the general formula (I):

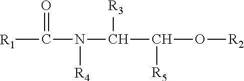

in which:
R₁ denotes:
either a saturated or unsaturated, linear or branched, C₁-C₅₀, hydrocarbon radical, preferably a C₅-C₅₀ hydrocarbon radical, it being possible for this radical to be substituted with one or more hydroxyl groups optionally esterified by an acid R₇COOH, R₇ being an optionally mono- or polyhydroxylated, linear or branched, saturated or unsaturated, C₁-C₃₅ hydrocarbon radical, it being possible for the hydroxyl(s) of the R₇ radical to be esterified by an optionally mono- or polyhydroxylated, linear or branched, saturated or unsaturated, C₁-C₃₅ fatty acid;
or a radical R″—(NR—CO)—R′, R denotes a hydrogen atom or a mono or polyhydroxylated, preferably monohydroxylated, C₁-C₂₀ hydrocarbon radical, R′ and R″ are independently hydrocarbon radicals of which the sum of the carbon atoms ranges from 9 to 30, R′ being a divalent radical;
or a radical R₈—O—CO—(CH₂)ₚ, R₈ denotes a C₁-C₂₀ hydrocarbon radical, p is an integer varying from 1 to 12;
R₂ denotes a hydrogen atom or a phosphorylethylamine or phosphorylethylammonium or a saccharide, preferably a (glycosyl)ₙ, (galactosyl)ₘ or sulphogalactosyl, radical, in which n is an integer varying from 1 to 4 and m is an integer varying from 1 to 8;
R₃ denotes a hydrogen atom or a hydroxylated or nonhydroxylated, saturated or unsaturated, C₁-C₃₃ hydrocarbon radical, it being possible for the hydroxyl(s) to be esterified by an inorganic acid or an acid R₇COOH, R₇ having the same meanings as above, it being possible for the hydroxyl(s) to be etherified by a saccharide radical, preferably a (glycosyl)ₙ, (galactosyl)ₘ or sulphogalactosyl radical, or a phosphorylethylamine or phosphorylethylammonium radical, it being also possible for R₃ to be substituted with one or more C₁-C₁₄ alkyl radicals;
   preferably, R₃ denotes a C₁₅-C₂₆α-hydroxyalkyl radical, the hydroxyl group being optionally esterified by a C₁₈-C₃₀α-hydroxy acid;
R₄ denotes a hydrogen atom, or a methyl or ethyl radical, or an optionally hydroxylated, linear or branched, saturated or unsaturated, C₃-C₅₀ hydrocarbon radical or a radical —CH₂—CHOH—CH₂—O—R₆ in which R₆ denotes a C₁₀-C₂₆ hydrocarbon radical or a radical R₈—O—CO—(CH₂)ₚ, R₈ denotes a C₁-C₂₀-hydrocarbon radical, p is an integer varying from 1 to 12,
R₅ denotes a hydrogen atom or an optionally mono- or polyhydroxylated, linear or branched, saturated or unsaturated, C₁-C₃₀ hydrocarbon radical, it being possible for the hydroxyl(s) to be etherified by a saccharide radical, preferably a (glycosyl)ₙ, (galactosyl)ₘ or sulphogalactosyl radical, or a phosphorylethylamine or phosphorylethylammonium radical;
   with the proviso that when R₃ and R₅ denote hydrogen or when R₃ denotes hydrogen and R₅ denotes methyl, then R₄ does not denote a hydrogen atom, or a methyl or ethyl radical.

Among the compounds of formula (I), the ceramides and/or glycoceramides whose structure is described by DOWNING in Journal of Lipid Research Vol. 35, 2060-2068, 1994, or those described in French patent application FR-2,673,179, whose teachings are incorporated herein by reference, are preferred.

The compounds of the ceramide type that are more particularly preferred according to the invention are the compounds of formula (I) for which R₁ denotes a saturated or unsaturated alkyl derived from optionally hydroxylated C₁₄-C₂₂ fatty acids; R₂ denotes a hydrogen atom; and R₃ denotes an optionally hydroxylated linear C₁₁₋₁₇, preferably C₁₃₋₁₅, radical

Such compounds are for example:
2-N-linoleoylamino-octadecane-1,3-diol,
2-N-oleolyamino-octadecane-1,3-diol,
2-N-palmitoylamino-octadecane-1,3-diol,
2-N-stearoylamino-octadecane-1,3-diol,
2-N-behenoylamino-octadecane-1,3-diol,
2-N-2-hydroxy-palmitoyl]-amino-octadecane-1,3-diol,
2-N-stearoylamino-octadecane-1,3,4-trio, preferably 2-N-stearoylamino-octadecane-1,3,4-triol is N-stearoylphytosphingosine, and
2-N-palmitoylamino-hexadecane-1,3-diol
or mixtures of these compounds.

Specific mixtures, such as, for example, mixtures of ceramide(s) 2 and ceramide(s) 5 according to the DOWNING classification, can also be used.

It is also possible to use the compounds of formula (I) for which R₁ denotes a saturated or unsaturated alkyl radical derived from fatty acids; R₂ denotes a galactosyl or sulphogalactosyl radical; and R₃ denotes a saturated or unsaturated C₁₂-C₂₂ hydrocarbon radical and preferably a group —CH═CH—(CH₂)₁₂—CH₃.

By way of example, there may be mentioned the product consisting of a mixture of glycoceramides, sold under the tradename GLYCOCER by the company WAITAKI INTERNATIONAL BIOSCIENCES.

It is also possible to use the compounds of formula (I) described in patent applications EP-A-0,227,994 and WO94/07844, which are incorporated herein by reference.

Such compounds include, for example, QUESTAMIDE H (bis-(N-hydroxyethyl-N-cetyl)malonamide) sold by the company QUEST, and cetylic acid N-(2-hydroxyethyl)-N-(3-cetyloxy-2-hydroxypropyl)amide.

It is also possible to use the N-docosanoyl-N-methyl-D-glucamine described in patent application WO94/24097, which is incorporated herein by reference.

The concentration of compounds of the ceramide type may vary from 0.0001% to 20% by weight approximately relative to the total weight of the composition, and preferably from 0.001 to 10% approximately and still more preferably from 0.005 to 3% by weight.

According to the invention, it is possible to use any fixing polymer known per se, which can be removed with shampoo, chosen from anionic, amphoteric, zwitterionic and nonionic polymers and mixtures thereof.

The fixing polymers may be used in a solubilized form or in the form of a latex or pseudolatex (aqueous dispersion of solid insoluble particles of polymer).

Thus, the anionic fixing polymers preferably used are polymers containing groups derived from carboxylic, sulphonic or phosphoric acid and have a molecular weight of from about 500 to 5,000,000.

These carboxylic groups are provided by unsaturated mono-, di-, or tricarboxylic acid monomers such as those corresponding to the formula (II):

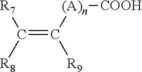

in which n is an integer from 0 to 10, A denotes a methylene group, optionally linked to the carbon atom of the unsaturated group or to the neighboring methylene group, when n is greater than 1, through a heteroatom such as oxygen or sulphur, R₇ denotes a hydrogen atom, or a phenyl or benzyl group, R₈ denotes a hydrogen atom, or a carboxyl or lower alkyl group, R₉ denotes a hydrogen atom or a lower alkyl group, a group —CH₂—COOH, or a phenyl or benzyl group.

In the abovementioned formula, a lower alkyl radical preferably denotes a group having 1 to 4 carbon atoms, and in particular methyl and ethyl.

The anionic polymers with carboxylic groups preferred according to the invention are:

A) the homo- or copolymers of acrylic or methacrylic acid or their salts and in particular the products sold under the names VERSICOL E or K by the company ALLIED COLLOID, ULTRAHOLD by the company BASF, the copolymers of acrylic acid and of acrylamide sold in the form of their sodium salt under the names RETEN 421, 423 or 425 by the company HERCULES, the sodium salts of the polyhydroxycarboxylic acids;
B) the copolymers of acrylic or methacrylic acids with a monoethylene monomer such as ethylene, styrene, vinyl esters, acrylic or methacrylic acid esters, optionally grafted onto a polyalkyleneglycol such as polyethyleneglycol and optionally crosslinked. Such polymers are described in particular in French patent 1,222,944 and German application 2,330,956, which are incorporated herein by reference, copolymers of this type containing in their chain an acrylamide unit optionally N-alkylated and/or hydroxyalkylated as described especially in Luxembourg patent applications 75370 and 75371, which are incorporated herein by reference, or offered under the name QUADRAMER by the company AMERICAN CYANAMID. There may also be mentioned the copolymers of acrylic acid and of C₁-C₄ alkylmethacrylate and the copolymer of methacrylic acid and of ethyl acrylate sold under the name LUVIMER MAEX by the company BASF;
C) the copolymers derived from crotonic acid such as those containing in their chain vinyl propionate or acetate units and optionally other monomers such as methallyl or allyl esters, vinyl ether or vinyl ester of a linear or branched saturated carboxylic acid with a long hydrocarbon chain, preferably those containing at least 5 carbon atoms, and more preferably those containing from 5 to 30 carbon atoms, it being possible for these polymers to be optionally grafted and crosslinked or alternatively a vinyl, allyl or methallyl ester of an α- or β-cyclic carboxylic acid. Such polymers are described, for example, in French patents 1,222,944; 1,580,545; 2,265,782; 2,265,781; 1,564,110 and 2,439,798, which are incorporated herein by reference. Commercial products entering into this class are the resins 28-29-30, 26-13-14 and 28-13-10 sold by the company NATIONAL STARCH;
D) the polymers derived from itaconic, fumaric or maleic acids or anhydrides with vinyl esters, vinyl ethers, vinyl halides, phenylvinyl derivatives, acrylic acid and its esters; these polymers may be esterified. Such polymers are described in particular in patents U.S. Pat. Nos. 2,047,398; 2,723,248; 2,102,113; and GB 839,805, which are incorporated herein by reference, and especially those sold under the names GANTREZ AN or ES by the company ISP. Polymers also entering into this class are the copolymers of maleic, citraconic and itaconic anhydrides and of an allyl or methallyl ester optionally containing an acrylamide or methacrylamide group, an α-olefin, acrylic or methacrylic esters, acrylic or methacrylic acids in their chain, the anhydride functional groups are monoesterified or monoamidated. These polymers are for example described in French patents 2,350,384 and 2,357,241, which are incorporated herein by reference.
E) The polyacrylamides containing carboxylate groups.

The polymers comprising sulphonic groups are polymers containing vinylsulphonic, styrenesulphonic, naphthalenesulphonic or acrylamidoalkylsulphonic units.

These polymers may be especially chosen from:
the salts of polyvinylsulphonic acid having a molecular weight of between about 1000 and 100,000 as well as the copolymers with an unsaturated comonomer such as acrylic or methacrylic acids and their esters as well as acrylamide or its derivatives, vinylethers and vinylpyrrolidone;
the salts of polystyrenesulphonic acid, the sodium salts having a molecular weight of about 500,000 and about 100,000 sold respectively under the names Flexan 500 and Flexan 130 by National Starch. These compounds are described in patent FR 2,198,719, which is incorporated herein by reference.
the salts of polyacrylamidesulphonic acids, those mentioned in patent U.S. Pat. No. 4,128,631, which is incorporated herein by reference, and more particularly polyacrylamidoethylpropanesulphonic acid sold under the name COSMEDIA POLYMER HSP 1180 by Henkel.

According to the invention, the anionic polymers are preferably chosen from the acrylic acid copolymers such as the terpolymer acrylic acid/ethylacrylate/N-tert-butylacrylamide sold under the name ULTRAHOLD STRONG by the company BASF, the copolymers derived from crotonic acid such as the terpolymers vinyl acetate/vinyl tert-butylbenzoate/crotonic acid and the terpolymers crotonic acid/vinyl acetate/vinyl neododecanoate sold under the name Resine 28-29-30 by the company NATIONAL STARCH, the polymers derived from itaconic, fumaric and maleic acids or anhydrides with vinyl esters, vinyl ethers, vinyl halides, phenylvinyl derivatives, acrylic acid and its esters such as the monoesterified maleic anhydride/methylvinyl ether copolymer sold under the name GANTREZ ES 425 by the company ISP, the copolymers of methacrylic acid and of methylmethacrylate sold under the name EUDRAGIT L by the company ROHM PHARMA, the copolymer of methacrylic acid and of ethylacrylate sold under the name LUVIMER MAEX by the company BASF and the copolymer vinyl acetate/crotonic acid sold under the name LUVISET CA 66 by the company BASF and the terpolymer vinyl acetate/crotonic acid/polyethyleneglycol under the name ARISTOFLEX A by the company BASF.

The anionic polymers most particularly preferred are those chosen from the monoesterified maleic anhydride/methylvinyl ether copolymer sold under the name GANTREZ ES 425 by the company ISP, the terpolymer acrylic acid/ethyl acrylate/N-tert-butylacrylamide sold under the name ULTRAHOLD STRONG by the company BASF, the copolymers of methacrylic acid and of methyl methacrylate sold under the name EUDRAGIT L by the company ROHM PHARMA, the terpolymers vinyl acetate/vinyl tert-butylbenzoate/crotonic acid and the terpolymers crotonic acid/vinyl acetate/vinyl neododecanoate sold under the name Resine 28-29-30 by the company NATIONAL STARCH, the copolymer of methacrylic acid and ethylacrylate sold under the name LUVIMER MAEX by the company BASF, the terpolymer vinylpyrrolidone/acrylic acid/lauryl methacrylate sold under the name ACRYLIDONE LM by the company ISP.

The amphoteric or zwitterionic polymers that can be used in accordance with the invention may be chosen from the polymers containing B and C units distributed statistically in the polymer chain where B denotes a unit which is derived from a monomer containing at least one basic nitrogen atom and C denotes a unit which is derived from an acidic monomer containing one or more carboxylic or sulphonic groups or alternatively B and C may denote groups which are derived from zwitterionic monomers of carboxybetaines or of sulphobetaines; B and C may also denote a cationic polymer chain containing primary, secondary, tertiary or quaternary amine groups, in which at least one of the amine groups carries a carboxylic or sulphonic group linked via a hydrocarbon radical or alternatively B and C form part of a chain of a polymer with an α,β-dicarboxylic ethylene unit in which one of the carboxylic groups has been caused to react with a polyamine containing one or more primary or secondary amine groups.

The amphoteric fixing polymers corresponding to the definition given above that are more particularly preferred are chosen from the following polymers:

1) The polymers resulting from the copolymerization of a monomer derived from a vinyl compound carrying a carboxylic group such as acrylic acid, methacrylic acid, maleic acid, alpha-chloroacrylic acid, and of a basic monomer derived from a substituted vinyl compound containing at least one basic atom such as the dialkylaminoalkylmethacrylate and acrylate, the dialkylaminoalkylmethacrylamide and acrylamide. Such compounds are described in U.S. Pat. No. 3,836,537, which is incorporated herein by reference.

(2) the polymers containing units which are derived from:

a) at least one monomer chosen from acrylamides or methacrylamides substituted on the nitrogen by an alkyl radical,

b) at least one acidic comonomer containing one or more reactive carboxylic groups, and

c) at least one basic comonomer such as esters with primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides preferred according to the invention are groups whose alkyl radicals contain from 2 to 12 carbon atoms and more preferably N-ethylacrylamide, N-tert-butylacrylamide, N-tert-octylacrylamide, N-octylacrylamide, N-decylacrylamide, N-dodecylacrylamide as well as the corresponding methacrylamides.

The acidic comonomers preferably are chosen from acrylic, methacrylic, crotonic, itaconic, maleic and fumaric acids as well as the alkyl monoesters having 1 to 4 carbon atoms of maleic or fumaric anhydrides or acids.

The preferred basic comonomers are methacrylates of aminoethyl, butylaminoethyl, N,N′-dimethylaminoethyl, N-tert-butylaminoethyl.

Particularly preferred are the copolymers whose CTFA name (4th ed. 1991) is octylacrylamide/acrylates/butylaminoethylmethacrylate copolymer, such as the products sold under the name AMPHOMER or LOVOCRYL 47 by the company NATIONAL STARCH.

(3) The partially or completely alkylated and crosslinked polyaminoamides derived from polyaminoamides of general formula (III):

CO—R₁₀—CO—Z

in which R₁₀ represents a divalent radical derived from a saturated dicarboxylic acid, a mono- or dicarboxylic aliphatic acid with ethylenic double bond, an ester of a lower alkanol having 1 to 6 carbon atoms and these acids or a radical which is derived from the addition of any one of the acids with a bis-primary or bis-secondary amine, and Z denotes a radical of a bis-primary, mono- or bis-secondary polyalkylene-polyamine and preferably represents:

a) in the proportions of 60 to 100 mol %, the radical (IV)

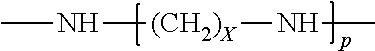

where x=2 and p=2 or 3, or x=3 and p=2 this radical being derived from the diethylenetriamine, triethylenetetraamine or dipropylenetriamine;

b) in the proportions of 0 to 40 mol %, the radical (IV) above, in which x=2 and p=1 and which is derived from ethylenediamine, or the radical which is derived from piperazine:

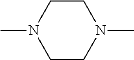

c) in the proportions of 0 to 20 mol %, the radical —NH—(CH₂)₆—NH— which is derived from hexamethylenediamine, these polyamino amines being crosslinked by adding a bifunctional crosslinking agent chosen from the epihalohydrins, diepoxides, dianhydrides, bis-unsaturated derivatives, by means of 0.025 to 0.35 mol of crosslinking agent per amine group of the polyamino amide and alkylated by the action of acrylic acid, chloroacetic acid or of an alkanesultone or of their salts.

The saturated carboxylic acids are preferably chosen from the acids having 6 to 10 carbon atoms such as adipic, 2,2,4-trimethyladipic and 2,4,4-trimethyladipic acid, terephthalic acid, the acids with ethylene double bond such as for example acrylic, methacrylic and itaconic acids.

The alkanesultones used in the alkylation are preferably propane or butanesultone, the salts of the alkylating agents are preferably the sodium or potassium salts.

4) The polymers containing zwitterionic units of formula (V):

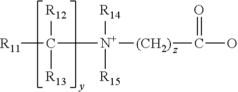

in which R₁₁ denotes a polymerizable unsaturated group such as an acrylate, methacrylate, acrylamide or methacrylamide group, y and z represent an integer from 1 to 3, R₁₂ and R₁₃ independently represent a hydrogen atom, methyl, ethyl or propyl, and R₁₄ and R₁₅ independently represent a hydrogen atom or an alkyl radical such that the sum of the carbon atoms in R₁₄ and R₁₅ does not exceed 10.

The polymers comprising such units may also comprise units derived from nonzwitterionic monomers such as dimethyl or diethylaminoethyl acrylate or methacrylate or alkyl acrylates or methacrylates, acrylamides or methacrylamides or vinyl acetate.

By way of example, there may be mentioned the copolymer of methylmethacrylate/methyl dimethylcarboxymethylammonioethylmethacrylate such as the product sold under the name DIAFORMER Z301 by the company SANDOZ.

(5) The polymers derived from chitosan containing monomeric units corresponding to the following formulae:

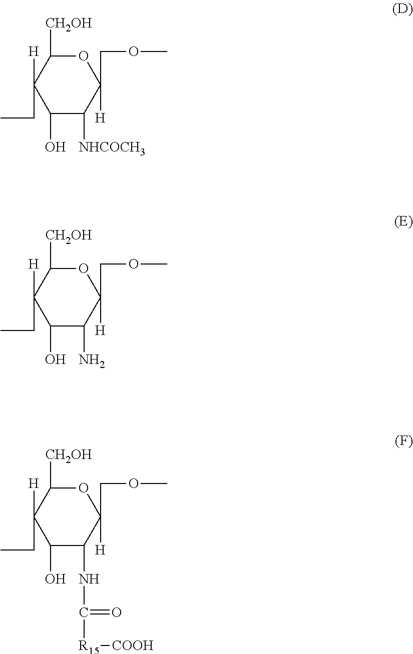

the D unit being present in proportions ranging from 0 to 30%, the E unit in proportions ranging from 5 to 50% and the F unit in proportions ranging from 30 to 90%, it being understood that in this F unit, R₁₆ represents a radical of formula:

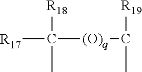

in which if q=0, R₁₇, R₁₈ and R₁₉, which are identical or different, each represent a hydrogen atom, a methyl, hydroxyl, acetoxy or amino residue, a monoalkylamine residue or a dialkylamine residue optionally interrupted by one or more nitrogen atoms and/or optionally substituted with one or more amine, hydroxyl, carboxyl, alkylthio or sulphonic groups, or an alkylthio residue whose alkyl group carries an amino residue, at least one of the R₁₇, R₁₈, and R₁₉ radicals being in this case a hydrogen atom; or if q=1, R₁₇, R₁₈, and R₁₉ each represent a hydrogen atom, as well as the salts formed by these compounds with bases or acids.

(6) The polymers derived from the N-carboxyalkylation of chitosan such as N-carboxymethyl chitosan or N-carboxybutyl chitosan sold under the name “EVALSAN” by the company JAN DEKKER.

(7) The polymers corresponding to the general formula (VI) are described in French patent 1,400,366:

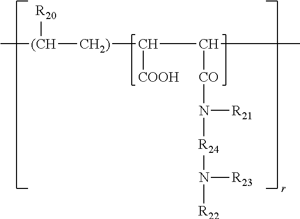

in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl, R₂₃ denotes a lower alkyl radical such as methyl or ethyl or a radical corresponding to the formula: —R₂₄—N(R₂₂)₂, R₂₄ representing a group —CH₂—CH₂—, —CH₂—CH₂—CH₂—, —CH₂—CH(CH₃)—, R₂₂ independently having the meanings mentioned above,
as well as the higher homologues of these radicals and containing up to 6 carbon atoms.

(8) Amphoteric polymers of the -D-X-D-X type chosen from:

a) the polymers obtained by the action of chloroacetic acid or sodium chioroacetate on the compounds containing at least one unit of formula:

-D-X-D-X-D- (VII)

where D denotes a radical

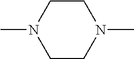

and X independently denotes the symbol E or E′, E or E′, which are identical or different, denote a bivalent radical which is an alkylene radical with a linear or branched chain containing up to 7 carbon atoms in the principal chain which is unsubstituted or substituted with hydroxyl groups and which may contain, in addition, oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, or hydroxyl, benzylamine, amine oxide, quaternary ammonium, amide, imide, alcohol, ester and/or urethane groups.

b) The polymers of formula:

-D-X-D-X- (VII′)

where D denotes a radical

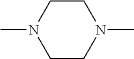

and X denotes the symbol E or E′ and at least one E′; E having the meaning indicated above and E′ is a bivalent radical which is an alkylene radical with a linear or branched chain having up to 7 carbon atoms in the principal chain, which is unsubstituted or substituted with one or more hydroxyl radicals and containing one or more nitrogen atoms, the nitrogen atom being substituted with an alkyl chain optionally interrupted by an oxygen atom and necessarily containing one or more carboxyl functional groups or one or more hydroxyl functional groups and betainized by reaction with chloroacetic acid or sodium chloroacetate.

(9) The copolymers (C₁-C₅)alkyl vinyl ethyer/maleic anhydride partially modified by semiamidation with an N,N-dialkylaminoalkylamine such as N,N-dimethylaminopropylamine or by semiesterification with an N,N-dialkanolamine. These copolymers may also contain other vinyl comonomers such as vinylcaprolactam.

The amphoteric polymers particularly preferred according to the invention are those of the family (3) such as the copolymers whose CTFA name is octylacrylamide/acrylates/butylaminoethylmethacrylate copolymer such as the products sold under the name AMPHOMER or LOVOCRYL 47 by the company NATIONAL STARCH.

The nonionic fixing polymers which can be used according to the present invention are chosen for example from:
the poly-β-alanines described more particularly in French patent No. 2,508,795, which is incorporated herein by reference;
the polyalkyloxazolines such as the polyethyloxazoline offered by the company DOW CHEMICAL under the names PEOX 50,000, PEOX 200,000 and PEOX 500,000;
the vinyl acetate homopolymers such as the product offered under the name APPRETAN EM by the company HOECHST or the product offered under the name RHODOPAS A 012 by the company RHONE POULENC;
the acrylic ester and vinyl acetate copolymers such as the product offered under the name RHODOPAS AD 310 from RHONE POULENC;
the ethylene and vinyl acetate copolymers such as the product offered under the name APPRETAN TV by the company HOECHST;
the copolymers of vinyl acetate and maleic ester, for example dibutyl maleate, such as the product offered under the name APPRETAN MB EXTRA by the company HOECHST;
the vinyl chloride homopolymers such as the products offered under the names GEON 460X45, GEON 460X46 and GEON 577 by the company GOODRICH;
the polyethylene waxes such as the products offered under the names AQUACER 513 and AQUACER 533 by the company BYK CERA;
the polyethylene/polytetrafluoroethylene waxes such as the products offered under the names DREWAX D-3750 by the company DRES AMEROID and WAX DISPERSION WD-1077 by the company R.T. NEWEY;
the maleic anhydride and polyethylene copolymers;
the homopolymers of alkyl acrylates and the homopolymers of alkyl methacrylates such as the product offered under the name MICROPEARL RQ 750 by the company MATSUMOTO or the product offered under the name LUHYDRAN A 848 S by the company BASF;
the copolymers of acrylic esters such as for example the copolymers of alkyl acrylates and alkyl methacrylates such as the products offered by the company ROHM & HAAS under the names PRIMAL ACZ 61 k and EUDRAGIT NE 30 D, by the company BASF under the names ACRONAL 601. LUHYDRAW LR 8833 or 8845, by the company HOECHST under the names APPRETAN N 9213 or N9212;
the copolymers of acrylonitrile and of a nonionic monomer chosen for example from butadiene and alkyl (meth)acrylates; there may be mentioned the products offered under the names NIPOL LX 531 B by the company NIPPON ZEON or those offered under the name CJ 0601 B by the company ROHM & HAAS
the styrene homopolymers such as the product RHODOPAS 5051 offered by the company RHONE POULENC;
the copolymers of styrene and of alkyl (meth)acrylate such as the products MOWILITH LDM 6911, MOWILITH DM 611 and MOWILITH LDM 6070 offered by the company HOECHST, the products RHODOPAS SD 215 and RHODOPAS DS 910 offered by the company RHONE POULENC, the product URAMUL SC 70 offered by the company DSM;
the copolymers of styrene, alkyl methacrylate and alkyl acrylate such as the product DAITISOL SPA offered by the company WACKHERR;
the copolymers of styrene and butadiene such as the products RHODOPAS SB 153 and RHODOPAS SB 012 offered by the company RHONE POULENC;
the copolymers of styrene, butadiene and vinylpyridine such as the products GOODRITE SB VINYLPYRIDINE 2528X10 and GOODRITE SB VINYLPYRIDINE 2508 offered by the company GOODRICH;
the polyurethanes such as the products offered under the names ACRYSOL RM 1020 and ACRYSOL RM 2020 by the company ROHM & HAAS, the products URAFLEX XP 401 UZ, URAFLEX XP 402 UZ by the company DSM RESINS;
the copolymers of alkyl acrylate and urethane such as the product 8538-33 by the company NATIONAL STARCH;
the polyamides such as the product ESTAPOR LO 11 offered by the company RHONE POULENC.

The alkyl radicals of the nonionic polymers preferably have from 1 to 6 carbon atoms.

According to the present invention, the fixing polymers are preferably anionic polymers.

The fixing polymer(s) is (are) preferably present in concentrations ranging from 0.01 to 20% by weight relative to the total weight of the composition, more preferably from 0.1 to 15% by weight and still more preferably from 0.5 to 10% by weight.

The cosmetically or dermatologically acceptable medium preferably consists of water or a mixture of water and cosmetically acceptable solvents such as monoalcohols, polyalcohols, glycol ethers or fatty acid esters, which may be used alone or as a mixture.

There may be mentioned more particularly lower alcohols such as ethanol, isopropanol, polyalcohols such as diethyleneglycol, glycol ethers, alkylethers of glycol or of diethyleneglycol.

The composition of the invention may also contain at least one additive chosen from thickeners, fatty acid esters, fatty acid esters and glycerol, silicones, perfumes, preservatives, sunscreens, proteins, vitamins, polymers, vegetable, animal, mineral or synthetic oils and any other additive conventionally used in the cosmetic field.

Preferably, the composition contains a silicone such as an oil, a resin, a wax or a silicone gum.

The compositions according to the invention may also contain one or more surfactants. The nature and the concentration of these surfactants are chosen by persons skilled in the art so as not to confer a detergent character on the composition. Preferably, the composition contains less than 4% by weight of anionic and/or amphoteric and/or zwitterionic detergent surfactants.

These additives are present in the composition according to the invention in proportions which may range from 0 to 20% by weight relative to the total weight of the composition. The precise quantity of each additive depends on its nature and is easily determined by persons skilled in the art.

Of course, a person skilled in the art will be careful to choose the possible compound(s) to add to the composition according to the invention such that the advantageous properties intrinsically linked to the composition in accordance with the invention are not, or not substantially, altered by the addition envisaged

In particular, the compositions according to the invention preferably comprise less than 10% by weight, relative to the total weight of the composition of fatty substances such as waxes, oils, paraffin, C₈-C₃, fatty acid esters. Thus, the keratinous fibres treated with the compositions according to the invention do not have a greasy feel or appearance, and the fixing power of the composition is not reduced. Preferably, the composition according to the invention does not, or does not substantially, contain a cationic surfactant.

The compositions according to the invention may be provided in the form of a gel, milk, cream, dispersion, lotion, thickened to a greater or lesser extent or foam.

The compositions according to the invention preferably are used as leave-in products especially for holding the hairstyle, shaping the hair or hair-styling.

They are more particularly hair-setting lotions, lotions for blow-drying, fixing compositions (lacquers) or hair-styling compositions. The lotions may be packaged in various forms, especially in vaporizers, pump dispensers or in aerosol containers in order to ensure application of the composition in vaporized form or in foam form. Such forms of packaging are indicated, for example, when it is desired to obtain a spray, a lacquer or a foam for fixing or treating the hair.

When the composition according to the invention is packaged in aerosol form in order to obtain an aerosol foam or a lacquer, it comprises at least one propelling agent, which is preferably chosen from volatile hydrocarbons such as n-butane, propane, isobutane, pentane, a chlorinated and/or fluorinated hydrocarbon and mixtures thereof. It is also possible to use, as propelling agent, carbon dioxide gas, nitrous oxide, dimethyl ether, nitrogen, compressed air and mixtures thereof.

The subject of the invention is also a nontherapeutic method of treating keratinous fibres such as human hair, consisting of applying to the keratinous fibers a composition as defined above.

The invention will now be illustrated more fully with the aid of the following examples that should not be considered as limiting it to the embodiments described. (In the text with follows, AS means Active Substance).

### EXAMPLES

### Example 1

A blow-drying lotion of the following composition was prepared:

| | | |
|---|---|---|
| N-oleoyldihydrosphingosine (ceramide) | 0.02 | g |
| Monoesterified maleic anhydride/methylvinyl | 1 | g AS |
| ether copolymer sold by the company ISP | | |
| under the name GANTREZ ES 425 (fixing | | |
| polymer) | | |
| Ethanol | 50 | g |
| Water | qs for 100 | g |

The composition was prepared at the time of use by mixing a portion A containing the ceramide and 10 g of ethanol, and a portion B containing the polymer, water and the remainder of the ethanol.

The composition was applied to hair that had been washed and drained and then a blow-drying was performed. The dried hair was sleek and soft and had good hair-styling properties. The hair was quite resistant to blow-drying.

### Example 2

A blow-drying lotion of the following composition was prepared:

| | | |
|---|---|---|
| N-oleoyldihydrosphingosine (ceramide) | 0.02 | g |
| Copolymer of methacryloyletnyr-N,N-dimethyl- | 1 | g AS |
| carboxymethylbetaine and of butyl methacrylate | | |
| sold in solution at 30% AS in ethanol under | | |
| the name DIAFORMER Z301 by the company | | |
| SANDOZ | | |
| Ethanol | 50 | g |
| Demineralized water | qs for 100 | g |

The composition was prepared and applied in the same manner as in Example 1. The dried hair was sleek and soft and had good hair-styling properties. The hair was quite resistant to blow-drying.

### Example 3

A blow-drying lotion of the following composition was prepared:

| | | |
|---|---|---|
| N-oleoyldihydrosphingosine (ceramide) | 0.02 | g |
| Monoesterified maleic anhydride/methylvinyl | 1 | g AS |
| ether copolymer sold by the company ISP | | |
| under the name GANTREZ ES 425 | | |
| Copolymer of hydroxyethyl cellulose and | 0.5 | g |
| diallyidimethylammonium chloride sold | | |
| under the trade name CELQUAT L200 by | | |
| the company | | |
| NATIONAL STARCH | | |
| Ethanol | 50 | g |
| Demineralized water | qs for 100 | g |

The composition was prepared and applied in the same manner as in Example 1. The dried hair was sleek and soft and had good hair-styling properties. The hair was quite resistant to blow-drying.

### Example 4

A blow-drying lotion of the following composition was prepared:

| | | |
|---|---|---|
| N-oleoyldihydrosphingosine (ceramide) | 0.02 | g |
| Terpolymer vinyl acetate/crotonic acid/ | 1 | g AS |
| polyethyleneglycol under the name | | |
| ARISTOFLEX A by the company BASF | | |
| Amodimethicone sold under the name | 0.5 | g AS |
| DC 929 by the company DOW | | |
| CORNING at 35% AS | | |
| Ethanol | 17.2 | g |
| Demineralized water | qs for 100 | g |

The composition was prepared and applied in the same manner as in Example 1. The dried hair was sleek and soft and had good hair-styling properties. The hair was quite resistant to blow-drying.

### Comparative Tests

Four compositions 1A, 2A, 3A and 4A were prepared having respectively the same composition as those of Examples 1, 2, 3 and 4, with the exception that the ceramide was removed in compositions 1A, 2A, 3A and 4A.

The mass of hair recovered after a blow-drying on a wig was compared for each pair of compositions. The greater the mass of broken hair, the less the composition protected the hair.

Each half-wig was washed beforehand with 6 ml of standard shampoo. After rinsing and drying with a sponge towel, 2.4 ml of the first product were applied on a half-wig with the aid of a pipette. A blow-drying was performed. Next, 2.4 ml of the second product were applied to the second half-wig, and a blow-drying was performed.

The blow-drying was performed by an experienced hairdresser with the aid of a Centaure 3940 brush and a Mega sprint bi-turbo 1500 hair dryer (setting 2 and 2). After each blow-drying, the hair remaining on the brush was recovered and weighed and the mass of hair was compared for each of the compositions tested.

The results are assembled in the table below:

Mass of Hair Recovered on Brush After Blow-Drying

| Compositions Tested | Invention | Comparative |
|---|---|---|
| 1 | 58.7 | |
| 1A | | 93.7 |
| 2 | 78.2 | |
| 2A | | 121.8 |
| 3 | 33.1 | |
| 3A | | 63.3 |
| 4 | 13.6 | |
| 4A | | 37.6 |

For each pair of compositions (1,1A), (2,2A), (3,3A), (4,4A), it was noted that the mass of hair recovered on the brush after the blow-drying was substantially reduced for the compositions according to the invention 1, 2, 3 and 4 containing the ceramide.

1. A nondetergent cosmetic composition for the treatment of a keratinous fiber comprising in a cosmetically acceptable medium, at least one fixing polymer selected from anionic, nonionic, zwitterionic and amphoteric fixing polymers and at least one ceramide,
wherein said composition does not contain a cationic surfactant, a vinylpyrrolidone polymer or a cationic polymer containing primary, secondary or tertiary amine or quaternary ammonium groups in the principal chain
and having a viscosity, at 1% by weight of active substance in water, of less than 15 mPas.

2. A composition according to claim 1, wherein said ceramide corresponds to the—formula (I): 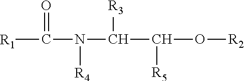
in which:
R₁ is
(1) a saturated or unsaturated, linear or branched, C₁-C₅₀ hydrocarbon radical, wherein said radical is optionally substituted with one or more hydroxyl groups that are optionally esterified by an acid R₇COOH, wherein R₇ is a linear or branched, saturated or unsaturated, C₁-C₃₅ hydrocarbon radical which is optionally mono- or polyhydroxylated by at least one hydroxyl, wherein said at least one hydroxyl of the R₇ radical is optionally esterified by an optionally mono- or polyhydroxylated, linear or branched, saturated or unsaturated, C₁-C₃₅ fatty acid;
(2) a radical R″—(NR—CO)—R′, wherein R denotes a hydrogen atom or a mono or polyhydroxylated C₁-C₂, hydrocarbon radical, R′ and R″ each independently denotes a hydrocarbon radical, wherein the sum of the carbon atoms of said hydrocarbon radicals R′ and R″ ranges from 9 to 30, and further wherein R′ is a divalent radical; or
(3) a radical R₈—O—CO—(CH₂)ₚ, wherein R denotes a C₁-C₂₀ hydrocarbon radical and p is an integer ranging from 1 to 12;
R₂ is selected from a hydrogen atom and phosphorylethylamine, phosphorylethylammonium, and saccharide radicals;
R₃ denotes a hydrogen atom or saturated or unsaturated, C₁-C₃₃ hydrocarbon radical which is optionally hydroxylated with at least one hydroxyl, wherein said at least one hydroxyl is optionally esterified by an inorganic acid or an acid R₇COOH, wherein R₇ is as defined above, and wherein the hydrogen atom of said at least one hydroxyl of the R₇ radical is optionally replaced with a radical selected from saccharide, phosphorylethylamine and phosphorylethylammonium radicals, and wherein R₃ is optionally substituted with one or more C₁-C₁₄ alkyl radicals;
R₄ denotes a hydrogen atom, or a methyl or ethyl radical, or an optionally hydroxylated, linear or branched, saturated or unsaturated, C₃-C₅₀ hydrocarbon radical or a radical
—CH₂—CHOH—CH₂—O—R₆ in which R₆ denotes a C₁₀-C₂₆ hydrocarbon radical, or a radical R₆—O—CO—(CH₂)ₚ in which R₈ denotes a C₁-C₂₀ hydrocarbon radical and p is an integer varying from 1 to 12,
R₅, denotes a hydrogen atom or a linear or branched, saturated or unsaturated, C₁-C₃₀ hydrocarbon radical which is optionally substituted with at least one hydroxyl, wherein the hydrogen atom of said at least one hydroxyl is optionally replaced with a radical selected from saccharide, phosphorylethylamine and phosphorylethylammonium radicals;
with the proviso that when R₃ denotes hydrogen and R₅ denotes hydrogen or a methyl radical, then R₄ is not a hydrogen atom or a methyl or ethyl radical.

3. A composition according to claim 2, wherein R denotes a monohydroxylated C₁-C₂₀ hydrocarbon radical.

4. A composition according to claim 1, wherein said at least one ceramide is selected from: 2-N-linoleoylamino-octadecane-1,3-diol, 2-N-oleolyamino-octadecane-1,3-diol, 2-N-paimitoylamino-octadecane-1,3-diol, 2-N-stearoylamino-octadecane-1,3-diol, 2-N-behenoylamino-octadecane-1,3-diol, 2-N-2-hydroxy-palmitoyl]-amino-octadecane-1,3-diol, 2-N-stearoylamino-octadecane-1,3,4-triol, and 2-N-paimitoylamino-hexadecane-1,3-diol.

5. A composition according to claim 1, wherein said at least one ceramide is selected from bis-(N-hydroxyethyl-N-cetyl)malonamide, N-(2-hydroxyethyl)-N-(3-cetyloxy-2-hydroxypropyl)amide of cetylic acid and N-docosandyl-N-methyl-D-glucamine.

6. A composition according to claim 1, wherein said anionic fixing polymers are selected from:
polymers containing carboxyl units derived from unsaturated mono-, di-, or tricarboxylic acid monomers of formula: 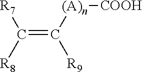
in which n is an integer from 0 to 10, A denotes a methylene group, optionally linked to the carbon atom of the unsaturated group or to the neighboring methylene group, when n is greater than 1, through a heteroatom such as oxygen or sulphur, R₇ denotes a hydrogen atom or a phenyl or benzyl group, R₈ denotes a hydrogen atom or a carboxyl or lower alkyl group, R₉ denotes a hydrogen atom or a lower alkyl group, a —CH₂—COOH group, or a phenyl or benzyl group; and
polymers comprising at least one unit derived from sulphonic acid monomers.

7. A composition according to claim 6, wherein said at least one unit derived from sulphonic acid monomers is selected from vinylsulphonic, styrenesulphonic, and acrylamindoalkylsulphonic units.

8. A composition according to claim 1, wherein said anionic fixing polymers are selected from:
A) homo- and copolymers of acrylic acid and salts thereof and homo and copolymers of methacrylic acid and salts thereof, and sodium salts of polyhydroxycarboxylic acids;
B) copolymers derived from crotonic acid, wherein said copolymers are optionally grafted, crosslinked, or grafted and crosslinked;
C) polymers derived from itaconic, fumaric or maleic acids or anhydrides thereof with vinyl esters, vinyl ethers, vinyl halides, phenylvinyl derivatives, or acrylic acid, wherein said anhydrides are monoesterified or monoamidated; and
D) polyacrylamides containing carboxylate groups.

9. A composition according to claim 8, wherein said copolymers derived from crotonic acid are selected from copolymers containing in their chain vinyl propionate or acetate units and optionally at least one other monomer.

10. A composition according to claim 9, wherein said at least one other monomer is selected from vinyl ethers and esters of a linear or branched saturated carboxylic acid with a hydrocarbon chain containing at least 5 carbon atoms.

11. A composition according to claim 1, wherein said anionic fixing polymers are selected from:
acrylic acid/ethyl acrylate/N-tert-butylacrylamide terpolymer;
vinyl acetate/vinyl tert-butylbenzoate/crotonic acid terpolymers;
crotonic acid/vinyl acetate/vinyl neododecanoate terpolymers;
monoesterified maleic anhydride/methylvinyl ether copolymers;
copolymers of methacrylic acid and of methylmethacrylate;
copolymer of methacrylic acid and of ethyl acrylate;
vinyl acetate/crotonic acid copolymer; and
vinyl acetate/crotonic acid/polyethyleneglycol terpolymer.

12. A composition according to claim 1, wherein said amphoteric fixing polymers are selected from the polymers containing the units derived from:
a) at least one monomer selected from acrylamides and methacrylamides, wherein said acrylamides and methacrylamides are substituted on the nitrogen by an alkyl radical,
b) at least one acidic comonomer comprising one or more reactive carboxyl groups, and
c) at least one basic comonomer.

13. A composition according to claim 12, wherein said at least one basic comonomer is an ester of acrylic and methacrylic acids containing substituents selected from primary, secondary, tertiary and quaternary amine substituents or a product of quaternization of dimethylaminoethyl methacrylate with dimethyl or diethyl sulphate.

14. A composition according to claim 1, wherein said amphoteric fixing polymers are selected from the copolymers whose CTFA name is octylacrylamide/acrylates/butylaminoethylmethacrylate copolymer.

15. A composition according to claim 1, wherein said nonionic fixing polymers are selected from:
poly-β-alanines;
polyalkyloxazolines;
vinyl acetate homopolymers;
acrylic ester and vinyl acetate copolymers;
ethylene and vinyl acetate copolymers;
copolymers of vinyl acetate and maleic ester;
vinyl chloride homopolymers; polyethylene waxes;
polyethylene/polytetrafluoroethylene waxes;
maleic anhydride and polyethylene copolymers;
homopolymers of alkyl acrylates and the homopolymers of alkyl methacrylates;
copolymers of acrylic esters;
copolymers of acrylonitrile and of a nonionic monomer;
styrene homopolymers;
copolymers of styrene and of alkyl (meth)acrylate;
copolymers of styrene, alkyl methacrylate and alkyl acrylate;
copolymers of styrene and butadiene;
copolymers of styrene, butadiene and vinylpyridine; and
copolymers of alkyl acrylate and urethane.

16. A composition according to claim 15, wherein said copolymers of acrylic esters are selected from copolymers of alkyl acrylates and alkyl methacrylates, and wherein said copolymers of acrylonitrile and of a nonionic monomer are selected from butadiene and alkyl (meth)acrylates.

17. A composition according to claim 1, wherein said at least one ceramide is present in a concentration ranging from 0.0001 to 20% by weight relative to the total weight of the composition.

18. A composition according to claim 17, where said at least one ceramide is present in a concentration ranging from 0.001 to 10% by weight.

19. A composition according to claim 18, wherein said at least one ceramide is present in a concentration ranging from 0.005 to 3% be weight.

20. A composition according to claim 1, wherein said at least one fixing polymer is present in a quantity ranging from 0.01 to 20% by weight relative to the total weight of the composition.

21. A composition according to claim 20, wherein said at least one fixing polymer is present in a quantity ranging from 0.1 to 15% by weight.

22. A composition according to claim 21, wherein said at least one fixing polymer is present in a quantity ranging from 0.5 to 10% by weight.

23. A composition according to claim 1, further comprising at least one additive selected from thickeners, fatty acid esters, fatty acid esters and glycerol, silicones, anionic, amphoteric, and/or zwitterionic surfactants, perfumes, preservatives, sunscreens, proteins, vitamins, polymers, vegetable, animal, mineral or synthetic oils and any other additive conventionally used in the cosmetic field.

24. A composition according to claim 1, wherein said cosmetically acceptable medium comprises water or a mixture of water and at least one cosmetically acceptable solvent.

25. A composition according to claim 24, wherein said at least one cosmetically acceptable solvent is selected from monoalcohols, polyalcohols, glycol ethers, and fatty acid esters.

26. A composition according to claim 1, wherein said composition is provided in the form of a gel, milk, cream, dispersion, lotion, which is thickened to a greater or lesser extent, or foam.

27. A composition according to claim 1, wherein said composition is a product for hair-styling, holding a hair-style or shaping hair.

28. A composition according to claim 1, wherein said composition is packaged in the form of a vaporizer or pump dispenser or in an aerosol container in order to obtain a spray, a lacquer or a foam.

29. A composition according to claim 1, wherein said at least one fixing polymer is solubilized in the cosmetically acceptable medium or is present in the form of an aqueous dispersion of insoluble solid particles.

30. A nontherapeutic method of treating a keratinous material comprising the step of applying to said material a composition according to claim 1.

31. A method according to claim 30, wherein said keratinous material is hair.

32. A method of protecting hair during blow-drying comprising the step of applying to said hair a composition according to claim 1.

33. A composition according claim 1, wherein said keratinous fiber is hair.

34. A composition according to claim 2, wherein said saccharide radicals defined in R₂, R₃, and R₅ are independently selected from (glycosyl)ₙ, (galactosyl)ₘ, and sulphogalactosyl radicals, wherein n is an integer ranging from 1 to 4 and m is an integer ranging from 1 to 8.

35. A composition according to claim 4, wherein said 2-N-stearoylamino-octadecane-1,3,4-triol is N-stearoylphytosphingosine.

36. A composition according to claim 8, wherein said copolymers of acrylic acid and salts thereof are selected from copolymers of acrylic acid and of acrylamide and salts thereof.

37. A composition according to claim 8, wherein said copolymers of acrylic acid and methacrylic acid are selected from copolymers of acrylic or methacrylic acids with at least one monoethylene monomer, said copolymers being optionally grafted onto a polyalkyleneglycol and optionally crosslinked, and wherein said copolymers of acrylic acid are further selected from copolymers of acrylic acid and of C₁-C₄ alkylmethacrylate.

38. A composition according to claim 1, wherein said anionic fixing polymers are selected from copolymers of maleic, citraconic or itaconic anhydrides and of an allyl or methallyl ester, wherein said copolymers optionally contain in their chain a group selected from acrylamides and methacrylamides, α-olefins, acrylic and methacrylic esters, and acrylic and methacrylic acids, wherein said anhydrides are monoesterified or monoamidated.

39. A composition according to claim 9, wherein said at least one other monomer is selected from methallyl and allyl esters.

40. A composition according to claim 37, wherein said at least one monoethylene monomer is selected from ethylene, styrene, vinyl esters, and acrylic and methacrylic acid esters.

41. A composition according to claim 8, wherein said copolymers of acrylic acid and methacrylic acid are selected from copolymers of acrylic or methacrylic acids with at least one monoethylene monomer, said copolymers being optionally grafted onto a polyalkyleneglycol and optionally crosslinked, and further wherein said copolymers of acrylic or methacrylic acids contain in their chain an acrylamide unit which is optionally N-alkylated and which is optionally hydroxyalkylated.

42. A composition according to claim 1, wherein said anionic fixing polymers are selected from polymers derived from itaconic, fumaric or maleic acids or anhydrides thereof with acrylic acid esters, wherein said anhydrides are monoesterified or monoamidated.

43. A composition according to claim 10, wherein said hydrocarbon chain contains from 5 to 30 carbon atoms.

44. A composition according to claim 2, wherein said hydrocarbon radical defined for R₁ is a C₅-C₅₀ hydrocarbon radical.
